(19) United States
(12) Patent Application Publication
(10) Pub. No.: US 2003/0109820 A1
(43) Pub. Date: Jun. 12, 2003

(54) Hemostatic agent delivery system

(76) Inventors: T. Daniel Gross, Los Gatos, CA (US); Dan J. Hammersmark, San Mateo, CA (US)

Correspondence Address: T. Gene Dillahunty, Esq., BURNS, DOANE, SWECKER & MATHIS, L.L.P., P.O. Box 1404, Alexandria, VA, 22313-1404, US

(21) Appl. No.: 10/013,584

(22) Filed: Dec. 7, 2001

(51) Int. Cl. A61F 13/20

(52) U.S. Cl. 604/11

(57) ABSTRACT

A hemostatic agent delivery system, comprising: a suture, pledget or introducer sheath coated or impregnated with a hemostatic agent. A hemostatic agent delivery system, comprising: an introducer sheath; a flow blocking system positioned at the distal end of a deployment device; and a hemostatic agent injection system. A hemostatic agent delivery system, comprising: an introducer sheath; and (a) a plug delivery shaft positionable within the introducer sheath, the plug delivery shaft dimensioned to push hemostatic material in the introducer sheath to a location adjacent to a vessel wall, or (b) a delivery sleeve receivable over the introducer sheath, delivery sheath dimensioned to push a cuff of hemostatic material to a location adjacent to a vessel wall. The hemostatic agent may comprises chitosan.

### TECHNICAL FIELD

The present invention relates in general to systems for achieving hemostasis at a cut or puncture in a vessel, and in particular to systems for closing an opening in the wall of a femoral artery following a catheterization procedure.

### BACKGROUND OF THE INVENTION

A number of diagnostic and interventional procedures require vascular access in which a catheter is introduced to the vascular system at a convenient access location and guided through the vascular system to a target location. When vascular access is no longer required, the catheter introducer sheath is removed and bleeding at the puncture site is stopped. Typically, such interventional catheterization procedures are performed by inserting the catheter through a puncture into the patient's femoral artery. Following such catheterization procedures, it is then necessary to close the hole or puncture into the femoral artery.

Various methods exist for closing such a puncture in the femoral artery. The primary method is the use of direct compression over the puncture to provide hemostasis (i.e. cessation of bleeding) until the patient's natural healing mechanisms form a blood clot at the puncture. To attain hemostasis, direct compression may need to be applied for a time period ranging from 20 minutes to as long as 90 minutes. Following the formation of a blood clot, the patient may be required to remain immobile for as long as 24 hours. Unfortunately, since such direct compression procedures rely upon clot formation, delays may be encountered since anticoagulants used in the vascular therapy process may need to first wear off. A further disadvantage of direct compression techniques to achieve hemostasis is that they are uncomfortable for the patient. Yet another disadvantage of direct compression techniques to achieve hemostasis is that they may result in occlusion of the blood vessel, thereby resulting in ischemia or thrombosis.

Other methods to close the puncture involve the use of sutures, plugs, staples, or combinations of these techniques. Such mechanical closure usually involves expensive devices, with technical complexity and associated operational learning curves.

### SUMMARY OF THE INVENTION

In various aspects, the present invention provides systems and methods for delivering a hemostatic agent to a cut or puncture at a vessel wall. A variety of delivery systems are contemplated, all keeping within the scope of the present invention.

In one aspect, the present invention provides a hemostatic agent delivery system comprising at least one suture carrying (e.g.: coated or impregnated with) a hemostatic agent. Preferably, the hemostatic agent comprises chitosan. Optionally, a tamper coated with a hemostatic agent may be used in association with the suture.

In another aspect, the present invention provides a hemostatic agent delivery system comprising a pledget which may be coated or impregnated with a hemostatic agent, and a system for positioning the pledget at the surface of a vessel. Again, the hemostatic agent preferably comprises chitosan.

In another aspect, the present invention provides a hemostatic agent delivery system comprising an introducer sheath coated with a hemostatic agent such as chitosan.

In another aspect, the present invention provides a hemostatic agent delivery system comprising an introducer sheath, an expandable diaphragm positioned at the distal end of a deployment device, and a hemostatic agent injection system adapted to deliver hemostatic agent through the introducer sheath. Preferably, the expandable diaphragm and deployment device are both dimensioned to be advanced through the introducer sheath. In optional aspects, the expandable diaphragm is inflatable and the deployment device may comprise an inflation tube. As an alternative to (or in addition to) an expandable diaphragm, an expandable balloon or anchor may also be used. In optional aspects, the hemostatic agent injection system comprises an injection syringe. Again, the hemostatic agent preferably comprises chitosan.

In another aspect, the present invention provides a hemostatic agent delivery system comprising an introducer sheath, and a plug delivery shaft which is slidably positionable within the introducer sheath, wherein the plug delivery shaft is dimensioned to push the plug of hemostatic material in the introducer sheath to a location adjacent to a vessel wall. The plug of hemostatic material preferably comprises a plug of chitosan, or a plug of bioabsorbable material coated or otherwise carrying chitosan. In optional aspects, the invention also comprises an arterial wall position detector based on tactile feedback or blood marking.

In another aspect, the present invention provides a hemostatic agent delivery system comprising an introducer sheath, and a delivery sleeve slidably receivable over the introducer sheath, wherein the delivery sleeve is dimensioned to push a cuff of hemostatic material received around the introducer sheath to a location adjacent to a vessel wall. Preferably, the cuff of hemostatic material comprises a cuff of chitosan, or a cuff of bioabsorbable material coated or otherwise carrying chitosan. In addition, the cuff is preferably sufficiently elastic such that it collapses inwardly sealing the passageway therethrough when the introducer sheath is removed therefrom.

In certain aspects of the invention, the hemostatic agent is also delivered along the subcutaneous tissue tract through which the vessel access sheath is placed. This is particularly advantageous in preventing “blood ooze” by sealing the minor blood vessels which may have been disrupted during dilation of the subcutaneous tissue.

### BRIEF DESCRIPTION OF THE DRAWINGS

FIG. 1 is an illustration of a suture closure according to an embodiment of the present invention.

FIG. 2 is an illustration of placement of a pledget according to an embodiment of the present invention.

FIG. 3 is an illustration similar to FIG. 1, but additionally showing a tamper used in association with the suture.

FIG. 4 is an illustration of an introducer sheath received into a patient's femoral artery, the introducer sheath being coated with a hemostatic agent.

FIGS. 5 to 7 illustrate successive steps in delivering a hemostatic agent to a vessel wall with an expandable diaphragm system, as follows:

FIG. 5 shows the expandable diaphragm being advanced through an introducer sheath.

FIG. 6A shows expansion of the diaphragm after it has been positioned intraluminally in the femoral artery. (FIG. 7A shows a system similar to that of FIG. 6A, but with an inflatable balloon substituted for the expandable diaphragm.)

FIG. 6B is an illustration of the delivery of the hemostatic agent to an area adjacent to the outer wall of a vessel after diaphragm has been expanded into position against the inner surface of the wall of the vessel. (FIG. 7B shows a system similar to that of FIG. 6B, but with an inflatable balloon substituted for the expandable diaphragm.)

FIGS. 8 to 11 illustrate successive steps in operating a system for delivering a plug of hemostatic material to a location adjacent to an outer wall of a cut or punctured vessel, as follows.

FIG. 8 shows the plug of hemostatic material being advanced through an introducer sheath.

FIG. 9 shows the plug of hemostatic material reaching the location adjacent to the outer wall of the vessel.

FIG. 10 shows the introducer sheath removed.

FIG. 11 shows the delivery shaft removed.

FIGS. 12 to 14 illustrate successive steps in operating a system for delivering a cuff of hemostatic material to a location adjacent to an outer wall of a cut or punctured vessel, as follows.

FIG. 12 shows the hemostatic material cuff positioned adjacent the outer wall of the vessel.

FIG. 13 shows the removal of the introducer sheath.

FIG. 14 shows the removal of the delivery sleeve.

FIGS. 15 and 16 show another system of delivering a hemostatic agent to a location adjacent the outer surface of a vessel having a cut out puncture therethrough, as follows.

FIG. 15 is a perspective view of the distal end of the device.

FIG. 16 is a cross sectional elevation view of the device, showing its operation.

### DETAILED DESCRIPTION OF THE DRAWINGS

The present invention provides a variety of novel systems for delivering suitable blood clotting or hemostatic agents to a location at, or near, the wall of an artery or other vessel having a cut or puncture therethrough. Accordingly, the present invention may complement any other existing cut, puncture, or wound closure system. The present invention is thus ideally suited to assist in sealing the hole in a femoral artery following a catheterization or anastomosis procedure.

The various aspects of the present invention provide systems which deliver the blood clotting or hemostatic agents to locations below the skin surface, thus achieving maximum hemostatic benefit at a location adjacent the cut or puncture through the vessel wall.

An advantage of the various embodiments of the present invention is that they serve to speed hemostasis during or after catherization of a vessel. Thus, the need to apply direct compression and immobilize a patient for an extended period of time after catheterization is minimized, or eliminated.

A further advantage of the various embodiments of the present invention is that they can be used to deliver a blood clotting or hemostatic agent to a surgical site on the outside of a vessel wall, while avoiding or preventing release of the blood clotting or hemostatic agent within the vessel.

A further advantage of the present invention is that it preferably uses “chitosan” which may be derived from “chitin”. “Chitin” is a polysaccharide that forms the ecoskeletons of insects and crustaceans. “Chitosan” is derived from chitin by deacetylation (i.e. removal of the acetic acid radical CH₃CO—. Chitosan has been found to offer excellent hemostatic benefits (i.e. assist in blood clot formation). It is believed clotting is assisted by the ionic interaction between the positively charged chitosan polymer and the negatively charged red blood cell membrane. An advantage of this is that such clotting mechanism operates independently of the normal blood coagulation cascade which results in fibrin formation. Thus, chitosan can advantageously be used in conjunction with blood treated with heparin (which inhibits fibrin formation). In addition, chitosan is biodegradable, with the advantage that it is eventually re-absorbed back into the body as a sugar.

It is to be understood that the hemostatic agent of the various embodiments may be in the form of a solid polymer, gel polymer or liquid. For example, when chitosan is used, it may be used in the form of a solid polymer, gel polymer or liquid.

It is to be understood, however, that although the most preferred aspects of the present invention use chitosan, the present invention is not so limited. Rather, any suitable hemostatic or blood clotting agent, including, but not limited to any form of chitin, can be used. In addition, other hemostatic materials such as fibrin and fibrinogen, may also be used instead of, or in addition to, the various presently contemplated chitins including chitosan.

A further advantage of embodiments of the present invention are that the may be used to percutaneously deliver the hemostatic or blood clotting agent to a location directly adjacent to the outer wall of the vessel. This is particularly advantageous in that the hemostatic agent is delivered to the exact location of the cut or puncture through the vessel wall.

A first aspect of the present invention is shown in FIG. 1 which illustrates a suture mediated closure.

A second aspect of the present invention is shown in FIG. 2 which illustrates placement of a medicated pledget.

FIG. 3 shows a tamper which is used in association with the knotted suture shown in either of FIGS. 1 or 2.

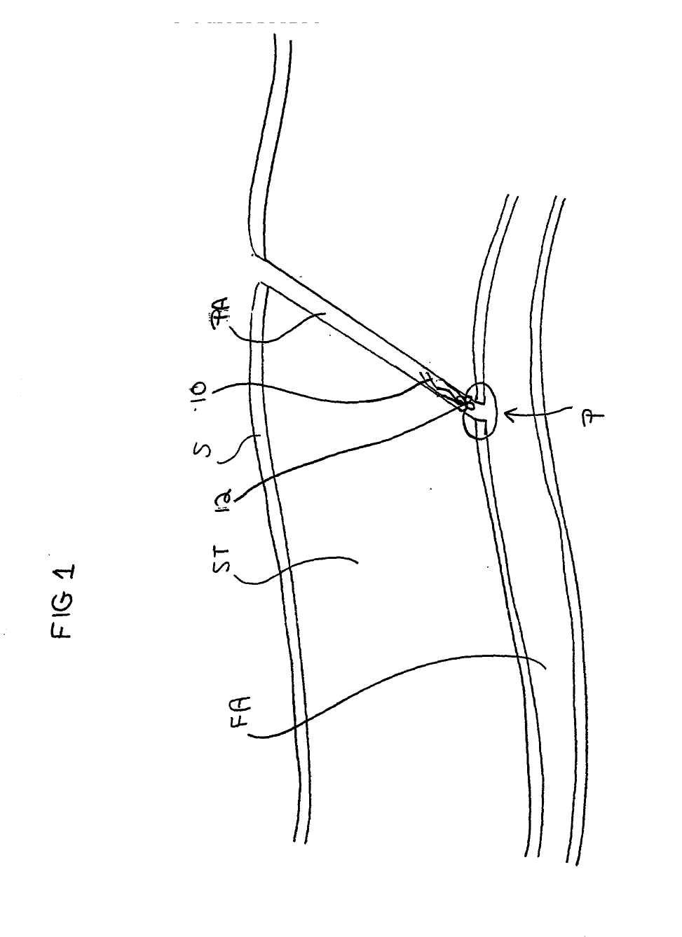

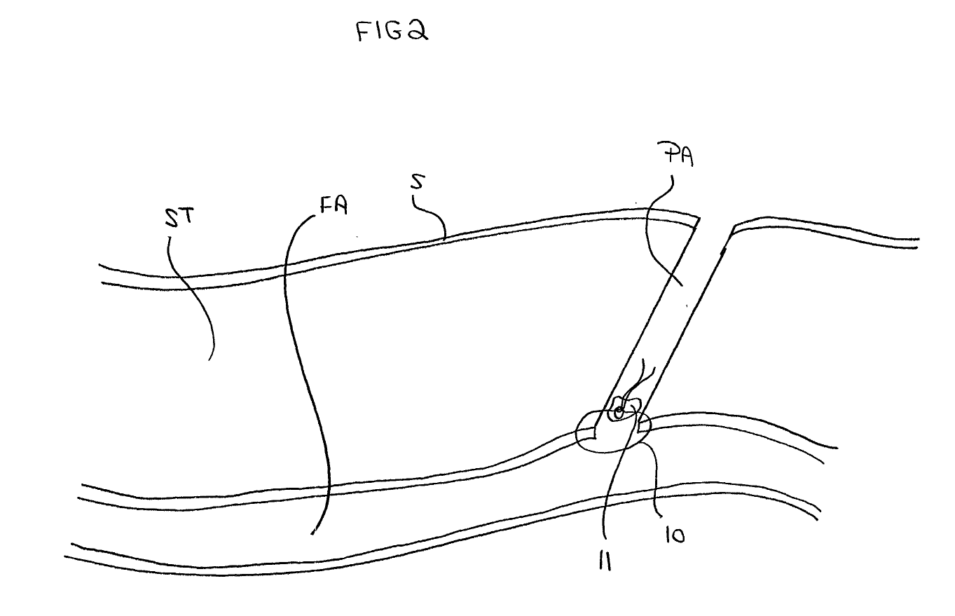

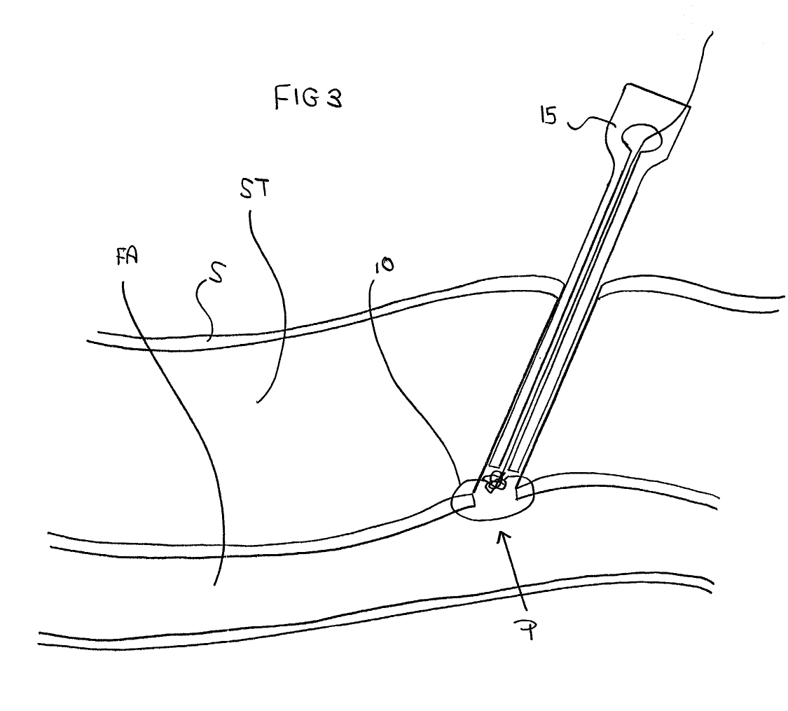

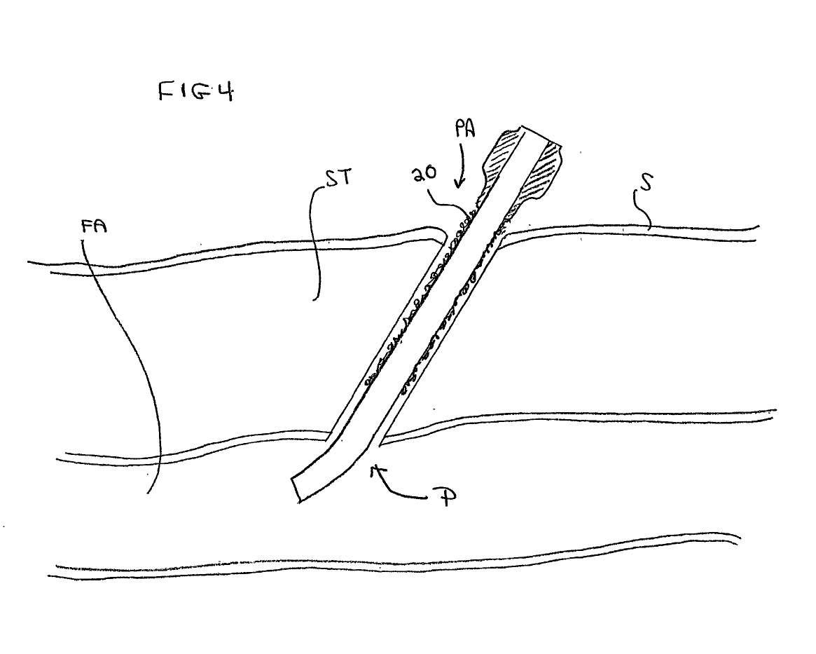

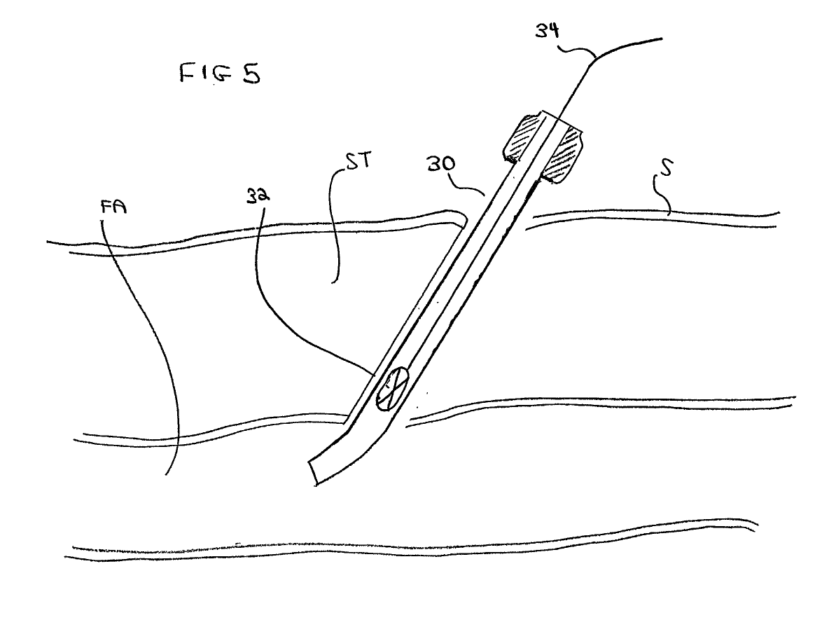

A third aspect of the present invention is shown in FIG. 4 which illustrates an introducer sheath coated with a hemostatic agent received into a patient's femoral artery.

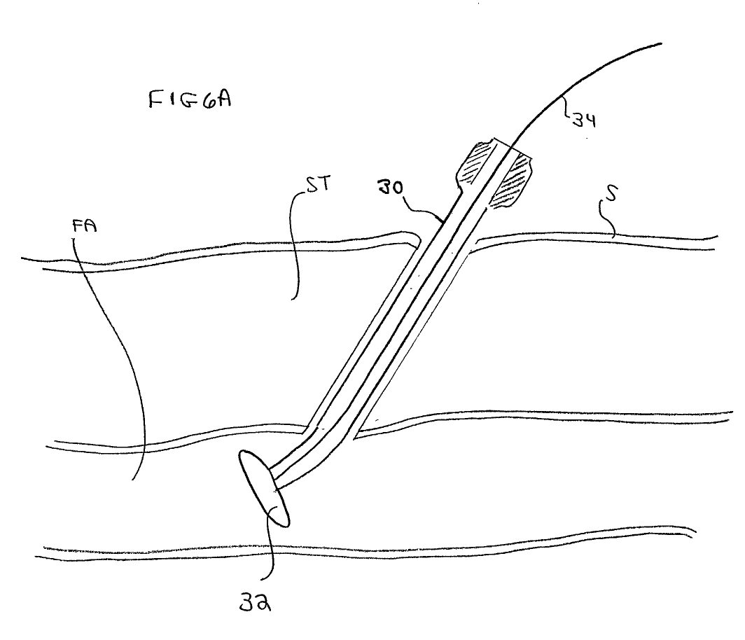

A fourth aspect of the present invention is shown in FIGS. 5 to 7 which illustrate successive steps in delivering a hemostatic agent to a vessel wall with an expandable diaphragm system.

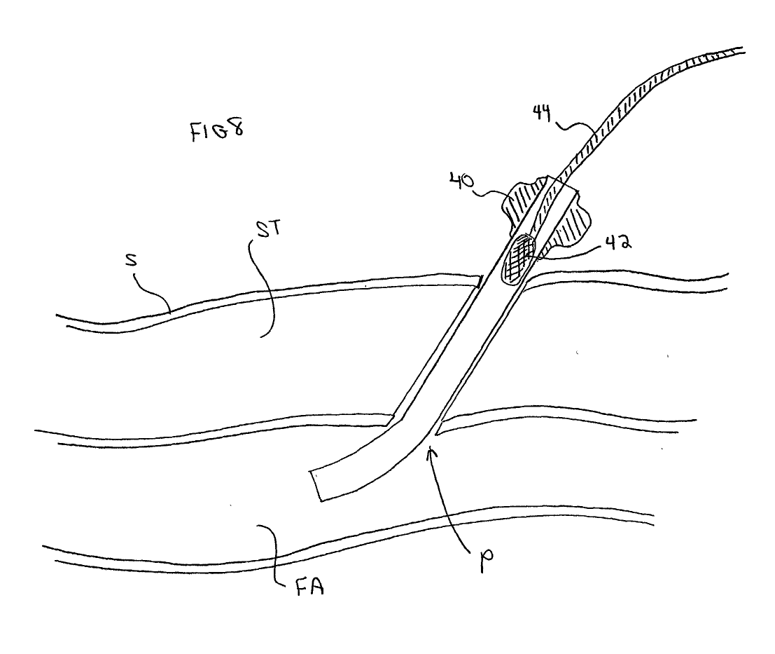

A fifth aspect of the present invention is shown in FIGS. 8 to 11 which illustrate successive steps delivering a plug of hemostatic material to a location adjacent to an outer wall of a cut or punctured vessel.

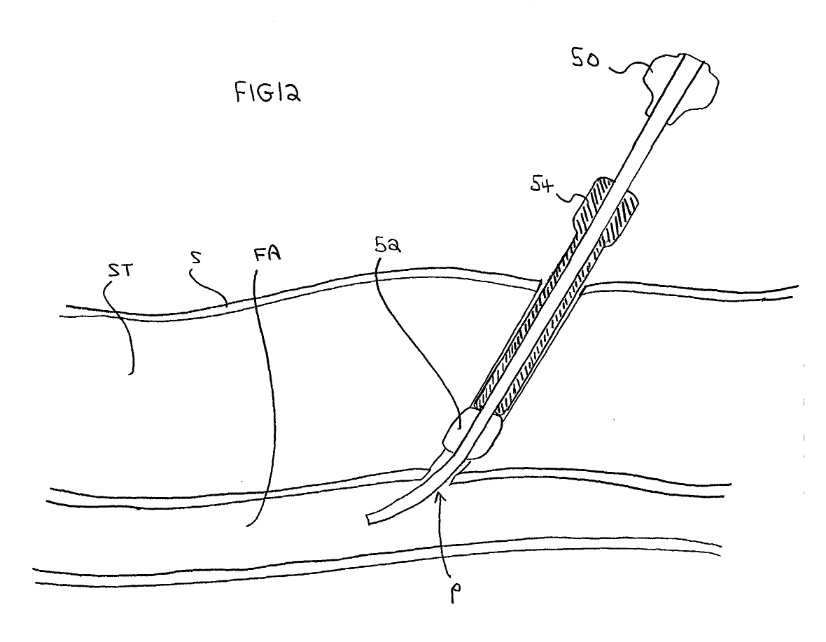

A sixth aspect of the present invention is shown in FIGS. 12 to 14 which illustrate successive steps in delivering a cuff of hemostatic material to a location adjacent to an outer wall of a cut or punctured vessel.

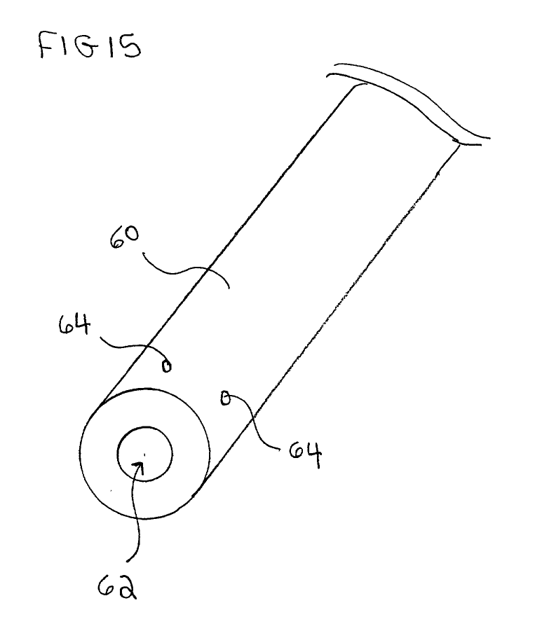

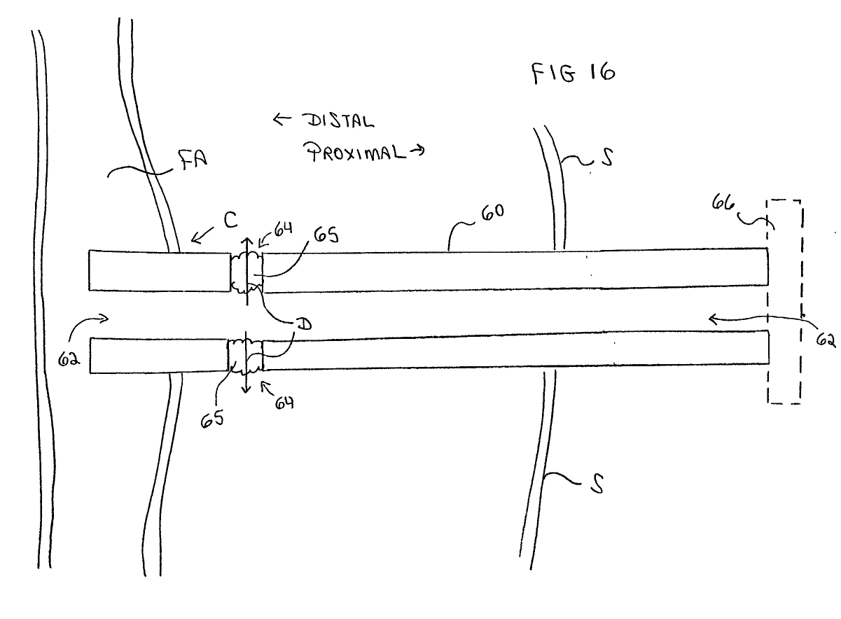

A seventh aspect of the present invention is shown in FIGS. 15 and 16 which illustrate a device which delivering hemostatic material placed in holes around the distal end of the device.

Referring first to FIG. 1, a patient's femoral artery FA is shown in cross section passing beneath their skin S and subcutaneous tissue ST. Femoral artery FA has been accessed by way of a percutaneous surgical procedure which has resulted in an arterial access site passing through puncture P in the wall of the artery. For example, puncture P in the wall of femoral artery FA may have resulted from inserting a catheter along path PA, through puncture P, and into the interior of femoral artery FA.

The present invention provides a variety of different systems and methods for promoting hemostasis adjacent puncture P in femoral artery FA. It is to be understood, however, that the present invention can be used to promote hemostasis adjacent any cut or puncture in the wall of any vessel, and that reference to puncture P through femoral artery FA is merely exemplary.

Moreover, the present systems can be used alone to percutaneously assist in sealing cuts or punctures in vessel walls, or can be used in conjunction with other vessel closure systems, such as suturing systems, colllagen plug systems and the like.

Returning to FIG. 1, a first aspect of the present invention simply comprises puncture P being sutured closed by suture 10. Preferably, suture 10 will be coated or infused with a hemostatic agent such as chitosan. Suture 10 may be placed by any suture closure device, and may be knotted by any existing suture knotting system. As knot 12 will be tied outside of femoral artery FA, a greater length of suture will be disposed on the exterior surface of C. Thus, a greater amount of hemostatic agent will be disposed on the exterior of puncture P. An advantage of having only a minimal length of suture 10 disposed within femoral artery FA is that only minimal amounts of the hemostatic agent will be released/activated within the interior of the vessel.

FIG. 2 is similar to FIG. 1, but shows a second aspect of the invention in which a pledget 11 has been tied into place by suture 10. In this second aspect of the invention, pledget 11 is preferably coated, impregnated, or otherwise carrying a hemostatic agent, such as chitosan. (Suture 10 may, or may not be coated or impregnated with a hemostatic agent, such as chitosan as well).

FIG. 3 shows a tamper 15 wich may optionally be used in either of the first (FIG. 1) or second (FIG. 2) aspects of the invention. Tamper 15 is preferably received over suture 10, and is advanced toward puncture P. In optional aspects, tamper 15 may also be coated with a hemostatic agent (such as chitosan). In these optional aspects, the tamper could be positioned such that it remains in place for a period of time subsequent to positioning over suture 10. After tamper 15 is removed, a layer of hemostatic agent would be shed from its surface, and deposited along path P, thereby promoting hemostasis along path P. This would advantageously inhibit blood ooze out through path PA to the patient's skin surface.

FIG. 4 shows a third aspect of the invention in which an introducer sheath 20 is percutaneously inserted into femoral artery FA. Introducer sheath 20 is coated with a hemostatic agent such as chitosan. Introducer sheath 20 is preferably dimensioned such that various surgical instruments (such as a catheter system) can be passed therethrough. After the surgical procedure is completed, (e.g.: after a catheter placed into the femoral artery has been removed), introducer sheath 20 is also removed. The layer of hemostatic agent on the outer surface of introducer sheath 20 would be shed from its surface, and thus be deposited along path PA, thereby promoting hemostasis along path PA. This would advantageously inhibit blood ooze out through path PA to the patient's skin surface. Moreover, the layer of hemostatic agent on the outer surface of introducer sheath 20 would also reduce blood ooze around introducer sheath 20 during the surgical procedure.

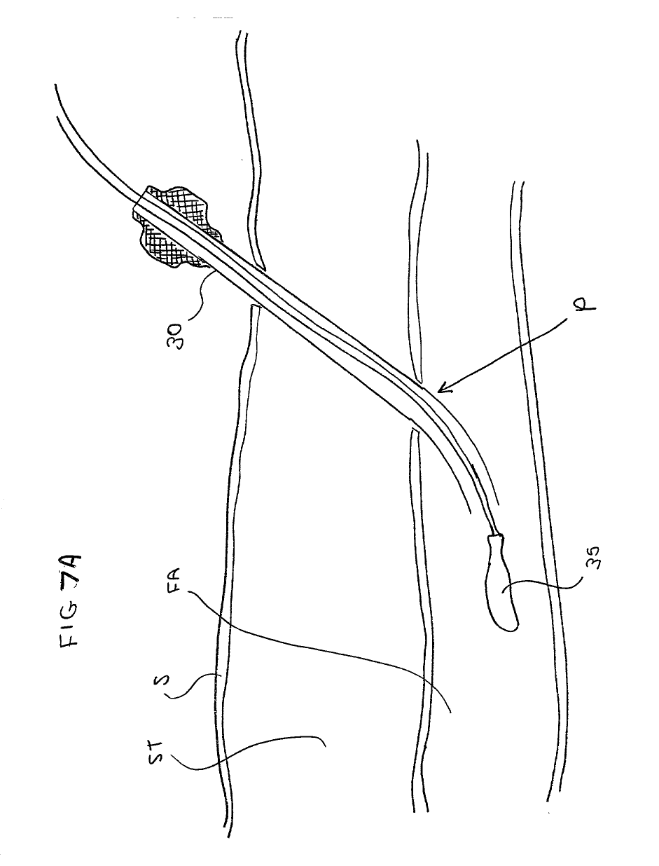

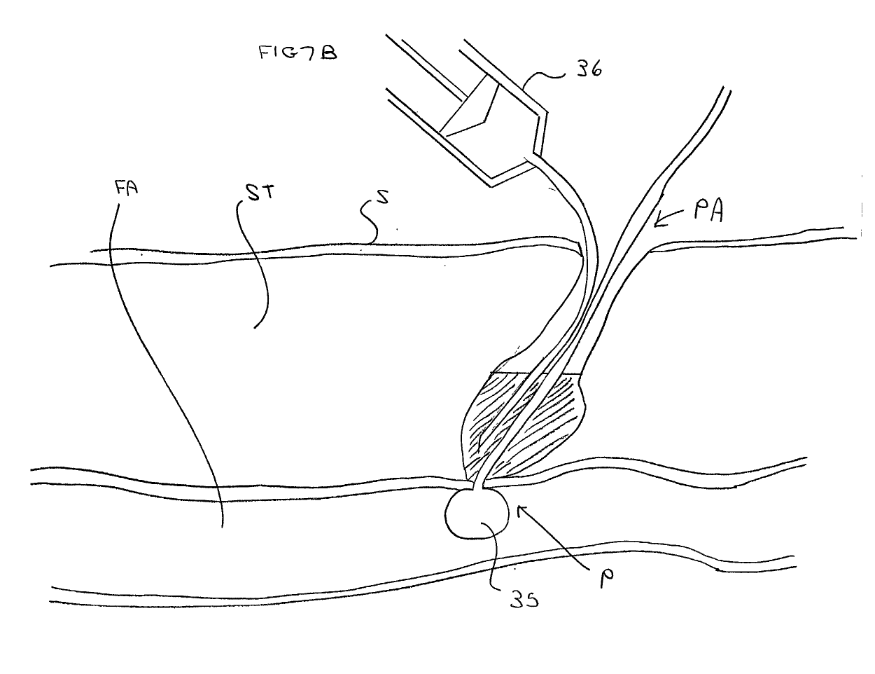

FIGS. 5, 6 and 7 show sequential steps in the operation of a fourth aspect of the present invention. An introducer sheath 30 is percutaneously introduced into femoral artery FA through puncture P. Introducer sheath 30 may preferably be dimensioned such that various surgical instruments (such as a catheter system) can be passed therethrough. After the surgical procedure is completed, (e.g.: after a catheter placed into the femoral artery has been removed), an expandable diaphragm 32 is then advanced therethrough. Expandable diaphragm 32 may preferably comprise a polymer membrane supported by superelastic hoop of nickel-titanium wire. Preferably, expandable diaphragm 32 is housed in a storage tube (not shown) prior to deployment. Alternatively, an inflatable balloon 35 (see FIGS. 7A and 7B) may instead be positioned intraluminally to temporarily occlude the puncture P using an inflation tube. In another alternative embodiment, an anchor, which may be biodegradable, can be provided in place of, or in addition to, diaphragm 30 or balloon 35.

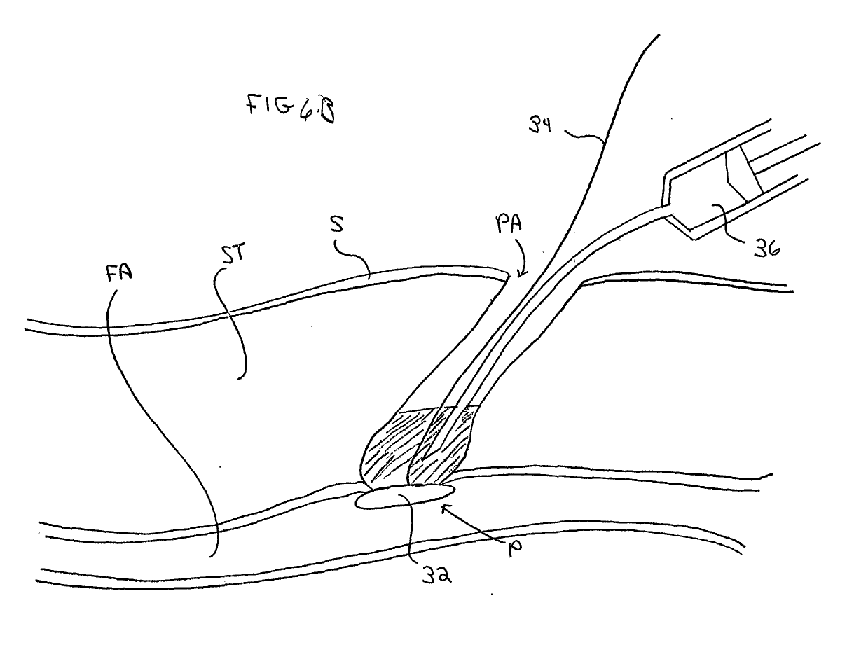

As shown in FIG. 5, expandable diaphragm 32 is advanced through introducer 30 until it reaches the position shown in FIG. 6A. At this time, expandable diaphragm 32 may be deployed by advancing it distally from the storage tube and then pulled back slightly such that expandable diaphragm 32 is positioned tightly against the inner surface of the arterial wall at puncture P as shown in FIG. 6B. Then, a hemostatic agent (for example, chitosan in liquid or gel form) can be injected by syringe 36 through path PA such that it collects or pools adjacent the exterior surface of the arterial wall at puncture P. As can be seen in FIG. 6B, the presence of expandable diaphragm 32 prevents the hemostatic agent from entering into femoral artery FA. After the hemostatic agent has been delivered, expandable diaphragm 32 can then be collapsed by drawing it proximally back into the storage tube and then removed from patient.

FIGS. 7A and 7B illustrate a system similar to FIGS. 6A and 6B, but instead introduce an inflatable balloon 35. As shown in FIG. 7A, balloon 35 is first advanced through introducer 30 into femoral artery FA. As shown in FIG. 7B, balloon 35 is then inflated and pulled back against the arterial wall at puncture P. Thus, balloon 35 operates to prevent a hemostatic agent (injected by syringe 36) from entering into femoral artery FA. Thus, balloon 35 operates in the same manner as expandable diaphram 32 of FIGS. 6A and 6B.

FIGS. 8, 9, 10 and 11 show sequential steps in the operation of a fifth aspect of the present invention. An introducer sheath 40 is percutaneously introduced into femoral artery FA through puncture P. Introducer sheath 40 may preferably be dimensioned such that various surgical instruments (such as a catheter system) can be passed therethrough. After the surgical procedure is completed, (e.g.: after a catheter placed into the femoral artery has been removed), a plug 42 of hemostatic agent is then pushed distally through introducer sheath 40 by a plug delivery shaft 44. In various aspects, plug 42 may comprise a plug of chitosan or a plug of bioabsorbable material coated or impregnated or otherwise carrying, chitosan. An advantage of using plug 42 is that it will assist in plugging or sealing path PA by its physical size (in addition to promoting hemostasis by its chemical composition).

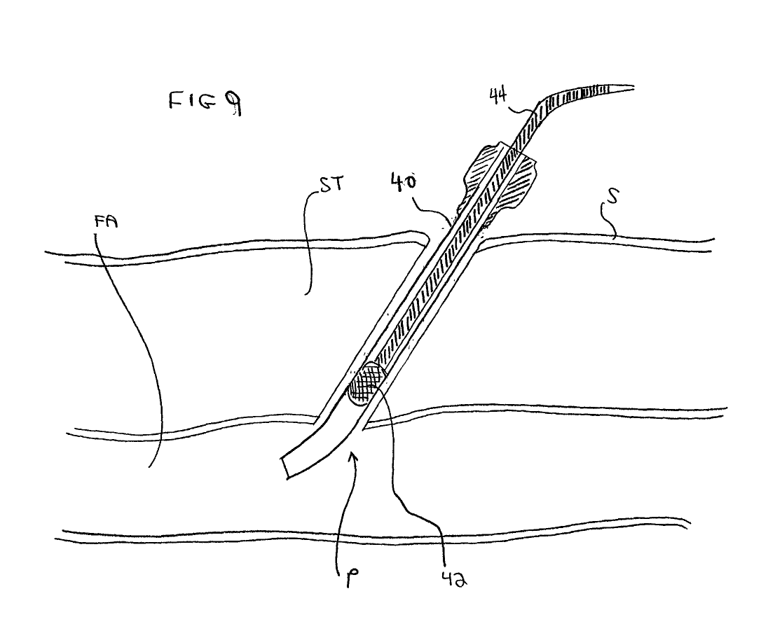

As shown in FIG. 9, plug 42 is preferably advanced to a location adjacent the outer surface of the arterial wall at puncture P. Such positioning is preferably assisted by a blood marking technique, or other depth gauge method determined using the initial arterial access needle and the introducer sheath.

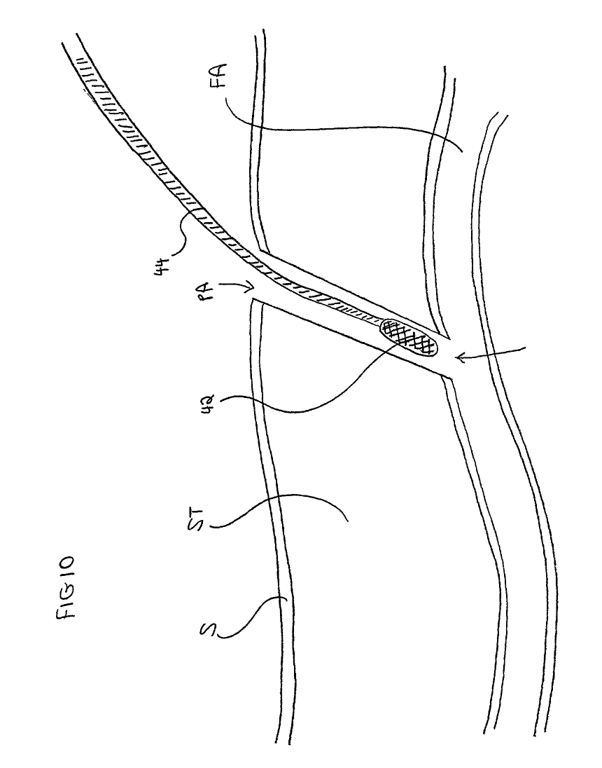

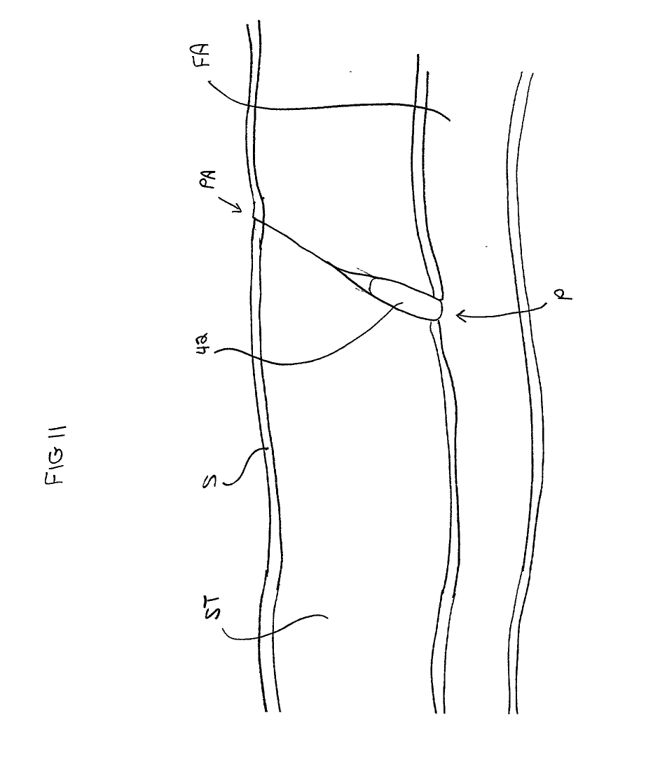

As shown in FIG. 10, introducer sheath 40 may then be removed. Thereafter, as shown in FIG. 11, plug delivery shaft 44 may also be removed. With the removal of delivery shaft 44, path PA will also start to collapse.

FIGS. 12, 13 and 14 show sequential steps in the operation of a sixth aspect of the present invention. An introducer sheath 50 is percutaneously introduced into femoral artery FA through puncture P. Introducer sheath 50 may preferably be dimensioned such that various surgical instruments (such as a catheter system) can be passed therethrough. After the surgical procedure is completed, (e.g.: after a catheter placed into the femoral artery has been removed), a cuff 52 of hemostatic agent is then pushed distally along the exterior of introducer sheath 40 by the distal end of a cuff delivery sleeve 54. In various aspects, cuff 52 may comprise a cuff of chitosan or a plug of bioabsorbable material coated with chitosan.

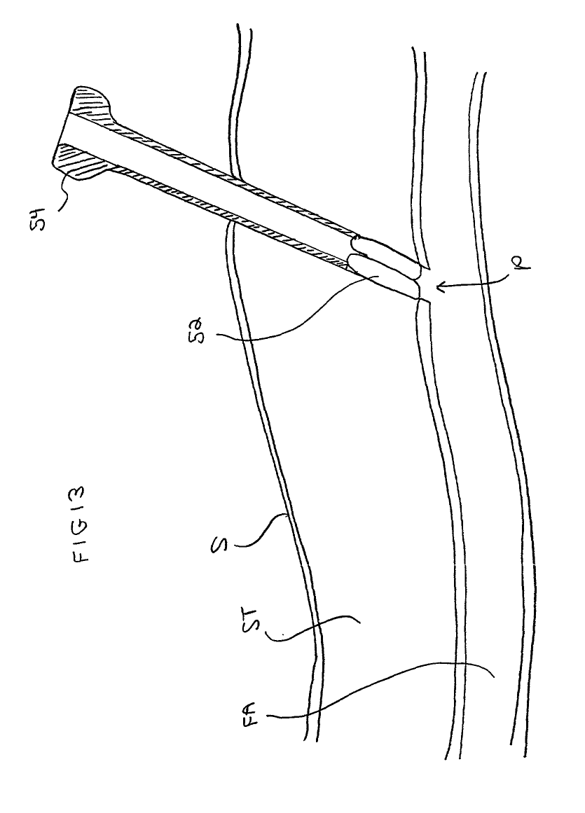

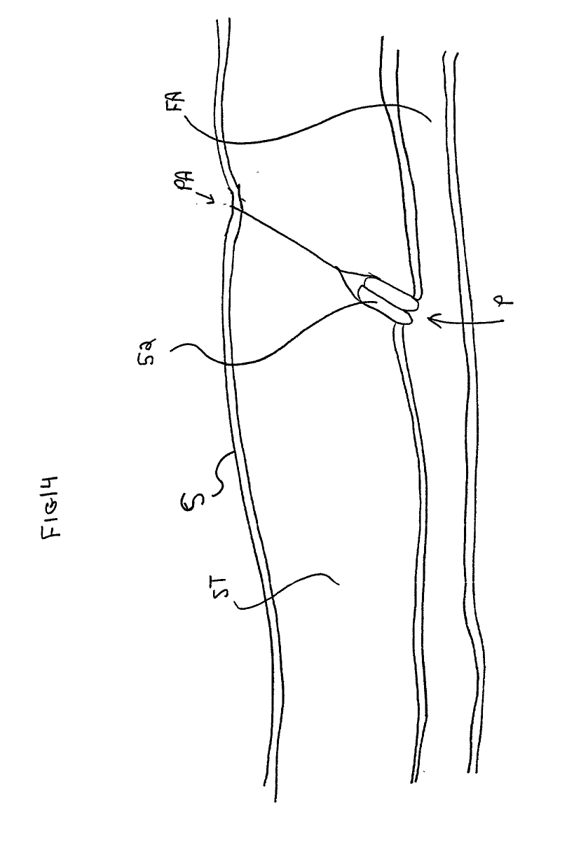

As shown in FIG. 13, introducer sheath 50 is then removed. Preferably, cuff 52 comprises a sufficiently elastic material such that it collapses when introducer sheath 50 has been removed. By collapsing, cuff 52 will assist in sealing path P, thereby preventing blood ooze up through path P, exiting through the patient's skin S. Lastly, as shown in FIG. 14, cuff delivery sleeve 54 is then removed, with path PA collapsing.

FIGS. 15 and 16 show alternate views of a seventh aspect of the present invention, as follows. FIG. 15 shows the distal end of a device 60 having a central bore 62, permitting catheter or other surgical access therethrough. A plurality of openings 64 pass outwardly from central bore 62 to the outer surface of device 60. Openings 64 may be disposed at any number of locations radially disposed around device 60.

As shown in FIG. 16, device 60 may be percutaneously inserted through a patient's skin S such that its distal end passes through puncture P and into femoral artery FA. Immediately when the distal end of device 60 enters femoral artery 60, blood will start to flow upwardly and start to exit from the proximal end of device 60. At this time, a cover 66 will be placed over the proximal end of device 60. Cover 66 may comprise any suitable closure system, including the thumb or finger of the operator. Concentrations of hemostatic agent 65 are stored in granular, pellet or gel form in openings 64. As soon as covering 66 is in place at the distal end of device 60, the pressure of the blood in femoral artery FA will cause the pressure within central bore 62 to increase. Thus, the concentrations of hemostatic agent 65 which are stored in granular, pellet or gel form in openings 64 will be forced outwardly in directions D. As can be seen, this will result in the hemostatic agent being disposed at locations which are adjacent to puncture P in the femoral artery. Moreover, as openings 64 are disposed some distance from the distal end of device 60, this will ensure that the concentrations of hemostatic agent 65 are delivered at locations which are exterior to femoral artery FA.

As stated herein, a preferred hemostatic agent for use in the various aspects of the present invention comprises chitin or chitosan. Although it is not so limited, the present invention comprises chitin or chitosan having any of the compositions as set forth in the following PCT publications by Struzczyk: WO/0187988, WO/0130855, WO/0119187, WO/0024785, WO/9834625, WO/9409192, WO/9406484, and WO/9403062. The present invention also comprises chitin or chitosan having any of the compositions as set forth in the following domestic and foreign patents and publications: U.S. Pat. Nos. 5,360,415; 5,204,107; 4,704,268; 4,699,135; 4,655,211; 4,651,725; 4,575,519; and 4,394,373; Japanese Patents JP63090507, JP60215003, and JP62167331, Norwegian Patent NO20013071, Australian Patent AU2591900, and PCT Publication WO0036918.

What is claimed is:

1. A hemostatic agent delivery system, comprising:
at least one suture coated with a hemostatic agent.

2. The system of claim 1, wherein the hemostatic agent comprises chitosan.

3. A hemostatic agent delivery system, comprising:
a pledget carrying a hemostatic agent; and
a device for positioning the pledget at the surface of a vessel.

4. The system of claim 3, wherein the hemostatic agent comprises chitosan.

5. The system of claim 3, wherein the system for positioning the pledget at the surface of the vessel comprises:
at least one suture.

6. A hemostatic agent delivery system, comprising:
an introducer sheath coated with a hemostatic agent.

7. The system of claim 6, wherein the hemostatic agent is chitosan.

8. The system of claim 6, wherein the introducer sheath is dimensioned to receive a suturing device therethrough.

9. The system of claim 6, wherein the hemostatic agent is releasably disposed on the introducer sheath.

10. A hemostatic agent delivery system, comprising:
an introducer sheath;
a flow blocking system positioned at the distal end of a deployment device, the flow blocking system and deployment device both being dimensioned to be advanced through the introducer sheath; and
a hemostatic agent injection system adapted to deliver hemostatic agent through the introducer sheath.

11. The system of claim 10, wherein the flow blocking system comprises an expandable diaphragm.

12. The system of claim 11, wherein the diaphragm is an expandable polymer membrane supported by a superelastic hoop.

13. The system of claim 12, further comprising:
a storage tube deployment device.

14. The system of claim 10, wherein the flow blocking system comprises an inflatable ballooon.

15. The system of claim 10, wherein the flow blocking system comprises a collagen plug.

16. The system of claim 10, wherein the hemostatic agent injection system comprises an injection syringe.

17. The system of claim 10, wherein the hemostatic agent comprises chitosan.

18. The system of claim 10, wherein the introducer sheath is dimensioned to receive a suturing device therethrough.

19. A hemostatic agent delivery system, comprising:
an introducer sheath; and
a plug delivery shaft which is slidably positionable within the introducer sheath, the plug delivery shaft being dimensioned to push a plug of hemostatic material in the introducer sheath to a location adjacent to a vessel wall.

20. The system of claim 19, wherein the plug of hemostatic material comprises a plug of chitosan.

21. The system of claim 19, wherein the plug of hemostatic material comprises a plug of bioabsorbable material carrying chitosan.

22. A hemostatic agent delivery system, comprising:
an introducer sheath; and
a delivery sleeve slidably receivable over the introducer sheath, delivery sleeve being dimensioned to push a cuff of hemostatic material received around the introducer sheath to a location adjacent to a vessel wall.

23. The system of claim 22, wherein the cuff of hemostatic material comprises a cuff of chitosan.

24. The system of claim 22, wherein the cuff of hemostatic material comprises a cuff of bioabsorbable material carrying chitosan.

25. The system of claim 22, wherein the cuff is sufficiently elastic to collapse inwardly when the introducer sheath is removed therefrom.

26. The system of claim 22, further comprising a hemostatic agent cuff disposed around the introducer sheath, wherein the delivery sleeve is positioned to abut a proximal end of the hemostatic agent cuff.

27. The system of claim 26, wherein the delivery sleeve holds the hemostatic agent cuff in position when the introducer sheath has been removed.

28. A hemostatic agent delivery system, comprising:
a tubular member having a longitudinal passageway therethrough, wherein the tubular member has at least one opening passing from the longitudinal passageway to the exterior surface of the device, and wherein the at least one opening is disposed near the distal end of the device.

29. The system of claim 28, further comprising:
hemostatic material disposed in the at least one opening.

30. The system of claim 29, wherein the hemostatic material comprises chitosan.

31. The system of claim 28, wherein the at least one opening comprises a plurality of openings disposed radially around the device.

32. The system of claim 28, further comprising:
a closure device positioned near the a proximal end of the device for selectively sealing the longitudinal passageway.

33. A method of delivering a hemostatic agent at a location adjacent a cut or puncture through a vessel wall, comprising:
percutaneously inserting an introducer sheath into the vessel;
advancing a fluid blocking system through the introducer sheath and into the vessel;
deploying the fluid blocking system;
positioning the fluid blocking system adjacent to an inner surface of the cut or puncture through the vessel wall;
injecting the hemostatic agent through the introducer sheath such that the hemostatic agent is disposed at a location adjacent an outer surface of the cut or puncture through the vessel wall.

34. The method of claim 33, wherein the fluid blocking system comprises an expandable diaphragm.

35. The method of claim 33, wherein the fluid blocking system comprises an inflatable balloon.

36. The method of claim 33, wherein the fluid blocking system comprises a collagen plug.

37. The method of claim 34, wherein the expandable diaphragm is advanced through the introducer sheath on the distal end of an inflation tube.

38. The method of claim 33, wherein the hemostatic agent comprises chitosan.

39. The method of claim 34, wherein the expandable diaphragm comprises an expandable polymer membrane supported by a superelastic hoop.

40. The method of claim 33, further comprising:
suturing closed the cut or puncture with a suturing device.

41. A method of assisting hemostasis at an arteriotomy in an arterial wall of a patient, the method comprising:
delivering chitosan into a subcutaneous tissue tract to a location adjacent the arteriotomy.

42. The method of claim 41, wherein the location adjacent the arteriotomy is at the arterial wall.

43. The method of claim 42, wherein the chitosan is carried by a pledget.

44. The method of claim 42, wherein the chitosan is carried by a collagen plug.

45. The method of claim 41, wherein the chitosan is carried by a suture.

46. The method of claim 41, wherein the chitosan is delivered by being shed from an outer surface of an introducer.

47. The method of claim 41, wherein the chitosan is delivered by injection after a puncture in the arterial wall has been sealed by a blocking device.

48. The method of claim 47, wherein sealing the arterial wall with the blocking device comprises:
expanding a diaphragm positioned adjacent to the arterial wall.

49. The method of claim 47, wherein sealing the arterial wall with the blocking device comprises:
inflating a balloon positioned adjacent to the arterial wall.

50. The method of claim 42, wherein a plug of the chitosan is introduced distally through an introducer.

51. The method of claim 42, wherein a cuff of the chitosan is introduced distally around an outer perimeter of an introducer.

52. The method of claim 42, wherein the chitosan is delivered by percutaneously inserting a tubular device having a longitudinal passageway extending therethrough into a patient such that a distal end of the device passes through a wall of the vessel, the tubular device having at least one opening passing from the longitudinal passageway to an external surface of the device, the at least one opening having a concentration of hemostatic material disposed therein; and
sealing the longitudinal passageway such that pressure in the vessel pushes the concentration of hemostatic material outwardly through the at least one opening to the location adjacent the cut or puncture through the vessel wall.
